(19) United States
(12) Patent Application Publication
(10) Pub. No.: US 2025/0188492 A1
(43) Pub. Date: Jun. 12, 2025

(54) Enhanced Transfer of Genetic Instructions to Effector Immune Cells

(71) Applicant: Janssen Biotech, Inc., Horsham, PA (US)

(72) Inventors: Madhusudhanan Sukumar, Spring House, PA (US); Suren RAJAKARUNA, Spring House, PA (US)

(21) Appl. No.: 18/997,683

(22) PCT Filed: Jul. 20, 2023
(86) PCT No.: PCT/IB2023/057424

§ 371 Date: Jan. 22, 2025

Related U.S. Application Data
(60) Provisional application No. 63/391,672, filed on Jul. 22, 2022.
(60) Provisional application No. 63/391,676, filed on Jul. 22, 2022.
(60) Provisional application No. 63/391,677, filed on Jul. 22, 2022.

(51) Int. Cl. C12N 15/86; C07K 14/725; C12N 5/0783

(52) U.S. Cl. CPC C12N 15/86; C07K 14/7051; C12N 5/0636; C12N 5/0646; C12N 2501/71; C12N 2501/727; C12N 2740/15043

(57) ABSTRACT

A method for preparing an immune effector cell for transduction, comprising a step for increasing the expression of low-density lipoprotein receptor (LDLR); and, optionally, a step for inhibiting intracellular anti-viral defense mechanisms of the immune effector cell.

### 1. CROSS-REFERENCE TO RELATED APPLICATIONS

This application claims the benefit of U.S. Provisional Patent Application No. 63/391,672, filed Jul. 22, 2022, U.S. Provisional Patent Application No. 63/391,676, filed Jul. 22, 2022, and U.S. Provisional Patent Application No. 63/391,677, filed Jul. 22, 2022. The entire contents of the aforementioned applications are incorporated herein by reference in their entirety.

### 2. FIELD

Provided herein, in some embodiments, are methods for producing bioengineered natural killer (NK) cells or T cells, e.g., NK cells or T cells expressing chimeric antigen receptors (CAR-NK cells or CAR-T cells), and uses thereof. Also provided herein, in some embodiments, is a composition comprising the CAR-NK cells or CAR-T cells and uses therefor treating a disease or disorder.

### 3. SUMMARY

In one aspect, provided herein is a method for preparing an immune effector cell for transduction, comprising a step for increasing the expression of low-density lipoprotein receptor (LDLR).

In some embodiments, the immune effector cell is autologous or allogeneic.

In some embodiments, the immune effector cell is a cytotoxic cell, and wherein optionally the cytotoxic cell is a natural killer cell.

In some embodiments, the immune effector cell is a gamma-delta T cell.

In some embodiments, the immune effector cell is an alpha-beta T cell.

In some embodiments, the step for increasing the expression of low-density lipoprotein receptor (LDLR) comprises contacting the immune effector cell with a statin.

In some embodiments, the statin is Rosuvastatin.

In some embodiments, the statin is Atorvastatin.

In some embodiments, the method further comprising a step for inhibiting intracellular anti-viral defense mechanisms of the immune effector cell.

In some embodiments, the step for inhibiting intracellular anti-viral defense mechanisms of the immune effector cell, comprising contacting the immune effector cell with an inhibitor of 3-phosphoinositide-dependent kinase 1 (PDK1).

In some embodiments, the inhibitor of PDK1 is BX795.

In some embodiments, the method further comprises providing one or more transduction enhancer(s).

In some embodiments, the transduction enhancer is Vectofusin.

In some embodiments, the transduction enhancer is Retronectin.

In some embodiments, the method further comprises introducing a nucleic acid into the immune effector cell.

In some embodiments, the nucleic acid comprises a promoter.

In some embodiments, the promoter is selected from a group consisting of CAG, PGK, EF1a, and EFS promoters.

In some embodiments, the nucleic acid is delivered via a lentiviral particle.

In some embodiments, the lentiviral particle is pseudotyped with vesicular stomatitis virus G (VSV-G).

In some embodiments, the nucleic acid encodes an exogenous functional receptor.

In some embodiments, the exogenous functional receptor is a chimeric antigen receptor (CAR) or a TCR.

In one aspect, provided herein is a method for preparing an immune effector cell for transduction, comprising contacting the immune effector cell with an agent that increases the expression of low-density lipoprotein receptor (LDLR).

In some embodiments, the immune effector cell is autologous or allogeneic.

In some embodiments, the immune effector cell is a cytotoxic cell, and wherein optionally the cytotoxic cell is a natural killer cell.

In some embodiments, the immune effector cell is a gamma delta T cell.

In some embodiments, the immune effector cell is an alpha beta T cell.

In some embodiments, the step for increasing the expression of low-density lipoprotein receptor (LDLR) comprises contacting the immune effector cell with a statin.

In some embodiments, the statin is Rosuvastatin.

In some embodiments, the statin is Atorvastatin.

In some embodiments, the method further comprises a step for inhibiting intracellular anti-viral defense mechanisms of the immune effector cell.

In some embodiments, the step for inhibiting intracellular anti-viral defense mechanisms of the immune effector cell, comprising contacting the immune effector cell with an inhibitor of 3-phosphoinositide-dependent kinase 1 (PDK1).

In some embodiments, the inhibitor of PDK1 is BX795.

In some embodiments, the method further comprises providing one or more transduction enhancer(s).

In some embodiments, the transduction enhancer is Vectofusin.

In some embodiments, the transduction enhancer is Retronectin.

In some embodiments, the method further comprises introducing a nucleic acid into the immune effector cell.

In some embodiments, the nucleic acid comprises a promoter.

In some embodiments, the promoter is selected from a group consisting of CAG, PGK, EF1a, and EFS promoters.

In some embodiments, the nucleic acid is delivered via a lentiviral particle.

In some embodiments, the lentiviral particle is pseudotyped with vesicular stomatitis virus G (VSV-G).

In some embodiments, the nucleic acid encodes an exogenous functional receptor.

In some embodiments, the exogenous functional receptor is a chimeric antigen receptor (CAR) or a TCR.

In one aspect, provided herein is a method for preparing an immune effector cell for transduction, comprising:
i. a first step for increasing the expression of low-density lipoprotein receptor (LDLR); and
ii. a second step for inhibiting intracellular anti-viral defense mechanisms of the immune effector cell;
wherein the first step can be conducted prior to, at the same time, or after the second step.

In some embodiments, the immune effector cell is autologous or allogeneic.

In some embodiments, the immune effector cell is a cytotoxic cell, and wherein optionally the cytotoxic cell is a natural killer cell.

In some embodiments, the method further comprises providing one or more transduction enhancer(s).

In some embodiments, the transduction enhancer is Vectofusin. In some embodiments, the transduction enhancer is Retronectin.

In some embodiments, the method further comprises introducing a nucleic acid into the immune effector cell. In some embodiments, the nucleic acid comprises a promoter. In some embodiments, the promoter is selected from a group consisting of CAG, PGK, EF1a, and EFS promoters.

In some embodiments, the nucleic acid is delivered via a lentiviral particle. In some embodiments, the lentiviral particle is pseudotyped with vesicular stomatitis virus G (VSV-G). In some embodiments, the nucleic acid encodes an exogenous functional receptor.

In some embodiments, the exogenous functional receptor is a chimeric antigen receptor (CAR) or a TCR.

In some embodiments, the first step comprises contacting the immune effector cell with a statin. In some embodiments, the statin is Rosuvastatin.

In some embodiments, the second step comprises contacting the immune effector cell with an inhibitor of 3-phosphoinositide-dependent kinase 1 (PDK1). In some embodiments, the inhibitor of PDK1 is BX795.

In another aspect, provided herein is a method for preparing an immune effector cell for transduction, comprising:
i. contacting the immune effector cell with a first agent that increases the expression of low-density lipoprotein receptor (LDLR);
ii. contacting the immune effector cell with a second agent that inhibits intracellular anti-viral defense mechanisms of the immune effector cell; and
iii. contacting the immune effector cell with a third agent that enhances transduction.

In yet another aspect, provided herein is a method comprising:
i. contacting an immune effector cell with a first agent that increases the expression of low-density lipoprotein receptor (LDLR);
ii. contacting the immune effector cell with a second agent that inhibits intracellular anti-viral defense mechanisms of the immune effector cell;
iii. contacting the immune effector cell with a third agent that enhances transduction; and
iv. introducing a nucleic acid into the immune effector cell.

### 4. BRIEF DESCRIPTION OF THE FIGURES

FIGS. 1A-1C show that CAR expression was downregulated over time in both NK-like cell lines and primary NK cells. FIG. 1A shows the down-regulation in NK-like cell line NK-92. FIG. 1B shows the down-regulation in NK-like cell line NK-L. FIG. 1C shows the down-regulation in primary NK cells.

FIGS. 2A-2C show that PDK1 inhibitor BX795 stabilized surface CAR expression in a dose dependent manner in primary NK cells. FIG. 2A shows the schematic schedule of the experiment. The experiment was run in duplicate (FIGS. 2B and 2C).

FIGS. 3A and 3B show that elevated expression of low-density lipoprotein receptor (LDLR) on primary NK cells was associated with increased Antigen-2 CAR expression. FIG. 3A shows the elevated expression of LDLR on primary NK cells after the cells were exposed to a statin, Rosuvastatin. FIG. 3B shows the increased Antigen-2 CAR expression associated with the elevated expression of LDLR.

FIG. 4 shows that the presence of transduction enhancers, Retronectin and Vectofusin, during the lentiviral transduction of primary NK cells was associated with increased Antigen-3 CAR expression.

FIGS. 5A-5D show that lentiviral transduction of primary NK cells in the presence of statin, BX795, Retronectin, and Vectofusin increased CAR stability on primary NK cells. The experiment was run in triplicate (FIGS. 5A-5C). FIG. 5D specifically shows the expression of Antigen-3 CAR on primary NK cells 14 days after the lentiviral transduction of primary NK cells under different conditions.

FIG. 6 shows that lentiviral transduction of primary αβ T cells in the presence of statin, BX795, Retronectin, and Vectofusin increases CAR expression.

FIG. 7 shows % viable primary γδ T cells following lentiviral transduction in the presence of enhancers: statin, BX795, and Vectofusin. LV: lentiviral vector; UTD: untransducted.

FIG. 8 shows % primary γδ T cells that were Antigen-4 CAR-positive, following lentiviral transduction in the presence of statin, BX795, and Vectofusin.

FIGS. 9A-9F show % primary γδ T cells that were CAR-positive, following lentiviral transduction in the presence of either atorvastatin (FIGS. 9A-9C) or rosuvastatin (FIGS. 9D-9F).

FIGS. 10A-10B show the LDLR expression shown as geometric mean, following lentiviral transduction in the presence of atorvastatin (FIG. 10A) or rosuvastatin (FIG. 10B) at concentrations indicated on the X-axis (μM).

FIGS. 11A-11B show frequency of Vγ9Vδ2 subset of primary γδ T cells following lentiviral transduction in the presence of statin, atorvastatin (FIG. 11A) or rosuvastatin (FIG. 11B), at concentrations indicated on the X-axis (μM).

### 5. DETAILED DESCRIPTION

Adoptive transfer of immune cells genetically modified to recognize malignancy-associated antigens is showing promise as a new approach of treating cancer (see, e.g., Brenner et al, Current Opinion in Immunology, 22 (2): 251-257 (2010); Rosenberg et al., Nature Reviews Cancer, 8 (4): 299-308 (2008)).

Genetically engineered T cells expressing chimeric antigen receptors (CARs) have emerged as one of the most powerful cancer treatments. In spite of the unprecedented treatment outcomes of CAR-T therapy, it is significantly limited by its autologous feature. For instant, T cells cannot be harvested from all of the patients, and the quality of T cells may not meet the standards of manufacturing. In addition, the conventional CAR-T therapy has intrinsic manufacturing issues, such as manufacturing failures, time delays, insufficient cell expansion, or heterogeneous products, which can be detrimental to the recipient patients. High costs are also unavoidable for such a highly personalized procedure.

Natural killer (NK) cells are part of the innate immune system and act as the first line of defense against cancer and viral infections. NK cells have gained increased attention as a promising alternative effector cell type for CAR engineering due to their rapid cytotoxicity against tumors, safety profile (low cytokine storm, minimal graft-versus-host disease, and low immune effector cell-associated neurotoxicity syndrome), and potential use as an off-the-shelf cellular therapy. However, optimizing highly efficient and clinically applicable gene transfer protocols for NK cells is challenging because NK cells are evolutionarily selected to have resistance against viral infection. Therefore, there is a need in the art of improved gene transfer protocols for NK cell-based immunotherapy.

The present disclosure is based, in part, on novel methods or processes for preparing an immune effector cell (e.g. NK cell) for transduction, and the uses thereof for making cellular therapies for treating a disease or disorder.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “immune effector cell” used herein refers to any of various types of cell that defends the body in an immune response by actively responding to a stimulus and effecting some change.

The term “cytotoxic cell” or “cytotoxic lymphocyte” used herein refers to certain subtypes of immune effector cell that kill their cognate infected or transformed targets through the cytotoxic granule exocytosis pathway. In some embodiments, the cytoxic cell is a natural killer cell (NK cell). In some embodiments, the cytoxic cell is cytotoxic T lymphocyte (CTL). In some embodiments, the cytoxic cell is alpha-beta (αβ) T cell. In some embodiments, the cytoxic cell is gamma-delta (γδ) T cell.

The term “natural killer cell” or “NK cell” used herein refers to a type of cytotoxic cell critical to the innate immune system. NK cells have the ability to recognize and kill stressed cells in the absence of antibodies and MHC, allowing for a much faster immune reaction.

The term “alpha-beta (αβ) T cells” used herein refers to a subset of T lymphocytes in the peripheral blood, that express αβ T cell receptors (TCRs).

The term “Gamma-delta (γδ) T cells” used herein refers to a subset of T lymphocytes in the peripheral blood, that express γδ T cell receptors (TCRs). In some embodiments, the γδ T cells comprise Vγ9Vδ2 subset of γδ T cells.

The term “cytotoxic T lymphocyte” or “CTL” used herein refers to a type of cytotoxic cell that destroys virus-infected cells, tumor cells, and tissue grafts that exist in the cytosol, or contiguous nuclear compartment. CTL is also known as CD8+ T cell as it expresses the CD8 glycoprotein at its surface and is associated with MHC class I molecules.

The term “low-density lipoprotein receptor” or “LDLR” refers to a class of cell surface proteins involved in receptor-mediated endocytosis of specific ligands. LDLR binds to particles called low-density lipoproteins (LDLs), which are the primary carriers of cholesterol in the blood.

The term “pseudotype” refers to produce a virus or viral vector in combination with a foreign viral envelope protein. The result is a pseudotyped virus that comprises an envelope protein originating from a different virus.

The term “vesicular stomatitis virus G” or “VSV-G” refers to a viral fusion protein of vesicular stomatitis virus (VSV). Viral fusion proteins are essential for the infection of enveloped virus. VSV-G mediates the fusion between virus envelope and host cellular membrane so that the viral genome is released into the host cell.

The term “antibody,” “immunoglobulin,” or “Ig” is used interchangeably herein, and is used in the broadest sense and specifically covers, for example, polyclonal or monoclonal antibodies (including agonist, antagonist, neutralizing antibodies, full length or intact monoclonal antibodies), antibody compositions with polyepitopic or monoepitopic specificity, monovalent antibodies, multivalent antibodies, or multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (koff) to association rate (kon) of a binding molecule (e.g., an antibody) to a monovalent antigen (koff/kon) is the dissociation constant KD, which is inversely related to affinity. The lower the KD value, the higher the affinity of the antibody. The value of KD varies for different complexes of antibody and antigen and depends on both kon and koff. The dissociation constant KD for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. Sec, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by FACS analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (KD) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1:755-68 (2006)). Also available for the preparation of human monoclonal antibodies are methods described in Cole et al., Monoclonal Antibodies and Cancer Therapy 77 (1985); Boerner et al., J. Immunol. 147 (1): 86-95 (1991); and van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6 (5): 561-66 (1995); Brüggemann and Taussing, Curr. Opin. Biotechnol. 8 (4): 455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27 (1): 55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26--H32 . . . 34 | H30--H35B | H27--H38 |
| | (Kabat | | | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single domain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Chimeric antigen receptor” or “CAR” as used herein refers to genetically engineered receptors, which can be used to graft one or more antigen specificity onto immune effector cells, such as T cells or NK cells. In some embodiments, the CAR comprises an extracellular antigen binding domain specific for one or more antigens (such as tumor antigens), a transmembrane domain, and an intracellular signaling domain of a NK cell and/or other receptors. “CAR-NK cell” refers to a NK cell that expresses a CAR.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single domain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a CAR described herein is a nucleic acid molecule that is identified and separated from at least one contaminant nucleic acid molecule with which it is ordinarily associated in the environment in which it was produced.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

As used herein, the term “allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “isolation” or “isolating” refers to a process of increasing the percentage of a certain substance in a composition. For example, isolating a type of cells from a population of cells refers to a process of creating a population of cells in which the percentage of this type of cells increases as compared to the percentage of this type of cells in the original population of cells. Therefore, the term “isolated” when used in the context of a type of cells does not mean that the isolated population of cells comprises 100% of this type of cells, rather it means the percentage of this type of cells increases in a population of cells after the isolation process.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. Sec, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Methods for Preparing Immune Effector Cells for Transduction

Provided herein, in one aspect, are methods for preparing immune effector cells for transduction.

In some embodiments, the immune effector cell is a cytotoxic cell. In certain embodiments, the cytotoxic cell is a natural killer cell. In certain embodiments, the cytotoxic cell is a cytotoxic T lymphocyte. In certain embodiments, the immune effector cell is an alpha beta T cell. In certain embodiments, the immune effector cell is a gamma delta T cell.

In some embodiments, the immune effector cell is contacted with one or more agent(s) that increases the expression of low-density lipoprotein receptor (LDLR). In some embodiments, the immune effector cell is contacted with one or more first agent(s) that increases the expression of low-density lipoprotein receptor (LDLR). In some embodiments, the immune effector cell is contacted with one or more second agent(s) that inhibits intracellular anti-viral defense mechanisms of the immune effector cell. In some embodiments, the immune effector cell is contacted with one or more third agent(s) that enhances transduction.

In some embodiments, the immune effector cell is contacted with one or more first agent(s) that increases the expression of low-density lipoprotein receptor (LDLR) and one or more second agent(s) that inhibits intracellular anti-viral defense mechanisms of the immune effector cell. In certain embodiments, the immune effector cell is contacted with the first agent(s) before being contacted with the second agent(s). In certain embodiments, the immune effector cell is contacted with the first agent(s) after being contacted with the second agent(s). In certain embodiments, the immune effector cell is contacted with the first agent(s) at the same time of being contacted with the second agent(s).

In some embodiments, the immune effector cell is contacted with one or more first agent(s) that increases the expression of low-density lipoprotein receptor (LDLR) and one or more third agent(s) that enhances transduction. In certain embodiments, the immune effector cell is contacted with the first agent(s) before being contacted with the third agent(s). In certain embodiments, the immune effector cell is contacted with the first agent(s) after being contacted with the third agent(s). In certain embodiments, the immune effector cell is contacted with the first agent(s) at the same time of being contacted with the third agent(s).

In some embodiments, the immune effector cell is contacted with one or more second agent(s) that inhibits intracellular anti-viral defense mechanisms of the immune effector cell and one or more third agent(s) that enhances transduction. In certain embodiments, the immune effector cell is contacted with the second agent(s) before being contacted with the third agent(s). In certain embodiments, the immune effector cell is contacted with the second agent(s) after being contacted with the third agent(s). In certain embodiments, the immune effector cell is contacted with the second agent(s) at the same time of being contacted with the third agent(s).

In some embodiments, the immune effector cell is contacted with one or more first agent(s) that increases the expression of low-density lipoprotein receptor (LDLR), one or more second agent(s) that inhibits intracellular anti-viral defense mechanisms of the immune effector cell, and one or more third agent(s) that enhances transduction. In certain embodiments, the immune effector cell is contacted with the first agent(s), then the second agent(s), then the third agent(s). In certain embodiments, the immune effector cell is contacted with the first agent(s), then the third agent(s), then the second agent(s). In certain embodiments, the immune effector cell is contacted with the second agent(s), then the first agent(s), then the third agent(s). In certain embodiments, the immune effector cell is contacted with the second agent(s), then the third agent(s), then the first agent(s). In certain embodiments, the immune effector cell is contacted with the third agent(s), then the first agent(s), then the second agent(s). In certain embodiments, the immune effector cell is contacted with the third agent(s), then the second agent(s), then the first agent(s). In certain embodiments, the immune effector cell is contacted with the first agent(s) before being contacted with the second agent(s) and third agent(s) at the same time. In certain embodiments, the immune effector cell is contacted with the first agent(s) after being contacted with the second agent(s) and third agent(s) at the same time. In certain embodiments, the immune effector cell is contacted with the second agent(s) before being contacted with the first agent(s) and third agent(s) at the same time. In certain embodiments, the immune effector cell is contacted with the second agent(s) after being contacted with the first agent(s) and third agent(s) at the same time. In certain embodiments, the immune effector cell is contacted with the third agent(s) before being contacted with the first agent(s) and second agent(s) at the same time. In certain embodiments, the immune effector cell is contacted with the third agent(s) after being contacted with the first agent(s) and second agent(s) at the same time. In certain embodiments, the immune effector cell is contacted with the first agent(s), second agent(s), and third agent(s) at the same time.

In certain embodiments, the first agent is a small molecule. In certain embodiments, the first agent is a big molecule. In certain embodiments, the first agent is organic. In certain embodiments, the first agent is inorganic. In certain embodiments, the first agent is a statin. In certain embodiments, the statin is Rosuvastatin. In certain embodiments, the statin is Atorvastatin. In certain embodiments, the statin is Fluvastatin. In certain embodiments, the statin is Lovastatin. In certain embodiments, the statin is Pitavastatin. In certain embodiments, the statin is Pravastatin. In certain embodiments, the statin is Simvastatin.

In certain embodiments, the second agent is an inhibitor of 3-phosphoinositide-dependent kinase 1 (PDK1). In certain embodiments, the inhibitor of PDK1 is a small molecule. In certain embodiments, the inhibitor of PDK1 is a big molecule. In certain embodiments, the inhibitor of PDK1 is organic. In certain embodiments, the inhibitor of PDK1 is inorganic. In certain embodiments, the inhibitor of PDK1 is BX795.

In certain embodiments, the third agent is a transduction enhancer. In certain embodiments, the third agent is a small molecule. In certain embodiments, the third agent is a big molecule. In certain embodiments, the third agent is organic. In certain embodiments, the third agent is inorganic. In certain embodiment, the third agent is Vectofusin. In certain embodiment, the third agent is Retronectin.

In certain embodiments, the immune effector cell is contacted with a first agent that increases the expression of low-density lipoprotein receptor (LDLR), a second agent that inhibits intracellular anti-viral defense mechanisms of the immune effector cell, and two third agents that enhance transduction. In certain embodiments, the first agent is Rosuvastatin. In certain embodiments, the second agent is BX795. In certain embodiments, the third agents are Vectofusin and Retronectin.

In some embodiments, the method further comprises transducing a nucleic acid into the immune effector cell. In certain embodiments, the nucleic acid is delivered via a viral particle. In certain embodiments, the nucleic acid is delivered via a lentiviral particle. In certain embodiments, the lentiviral particle is pseudotyped. In certain embodiments, the lentiviral particle is pseudotyped with vesicular stomatitis virus G (VSV-G).

In certain embodiments, the nucleic acid comprises a promoter. In certain embodiments, the promoter is selected from a group consisting of CAG, PGK, EF1a, and EFS promoters. In certain embodiments, the promoter is a CAG promoter. In certain embodiments, the promoter is a PGK promoter. In certain embodiments, the promoter is a EF1a promoter. In certain embodiments, the promoter is a EFS promoter.

In certain embodiments, the nucleic acid encodes an exogenous functional protein. In certain embodiments, the nucleic acid encodes an exogenous functional receptor. In certain embodiments, the exogenous functional receptor is a chimeric antigen receptor (CAR). In other embodiments, the exogenous function receptor is a TCR

### 5.3. Methods for Producing Immune Effector Cells Expressing Exogenous Functional Receptors

In yet another aspect, provided herein are method for producing immune effector cells expressing chimeric antigen receptors (CARs). Methods for preparing immune effector cells for transduction of a nucleic acid encoding a CAR is described in details in Section 4.2 above. In some embodiments, the immune effector cell is a cytotoxic cell. In certain embodiments, the cytotoxic cell is a natural killer cell. In certain embodiments, the cytotoxic cell is a cytotoxic T lymphocyte.

### 5.3.1. Chimeric Antigen Receptors

In some embodiments, the CAR provided herein comprises a polypeptide comprising: (a) an extracellular antigen binding domain; (b) a transmembrane domain; and (c) an intracellular signaling domain.

### Signal Peptide

In certain embodiments, the CARs provided herein may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor α, and IgG1 heavy chain.

### Extracellular Antigen Binding Domain

The extracellular antigen binding domain of the CARs described herein comprises one or more antigen binding domains. In some embodiments, the extracellular antigen binding domain of the CAR provided herein is mono-specific. In other embodiments, the extracellular antigen binding domain of the CAR provided herein is multispecific. In some embodiments, the extracellular antigen binding domain comprises two or more antigen binding domains which are fused to each other directly via peptide bonds, or via peptide linkers.

In some embodiments, the extracellular antigen binding domain comprises an antibody or a fragment thereof. For example, the binding domain may be derived from monoclonal antibodies (including agonist, antagonist, neutralizing antibodies, full length or intact monoclonal antibodies), antibody with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies). An antibody can be human, humanized, chimeric and/or affinity matured, as well as an antibody from other species, for example, mouse, rabbit, llama, etc. In some embodiments, the antibody include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

In a specific embodiment, the extracellular antigen binding domain of the present CARs comprise a single-chain Fv (sFv or scFv). ScFvs are antibody fragments that comprise the VH and VL antibody domains connected into a single polypeptide chain. Preferably, the scFv polypeptide further comprises a polypeptide linker between the VH and VL domains which enables the sFv to form the desired structure for antigen binding. See Pluckthun in The Pharmacology of Monoclonal Antibodies, vol. 113, Rosenburg and Moore eds., Springer-Verlag, New York, pp. 269-315 (1994).

In another specific embodiment, the extracellular antigen binding domain of the present CARs comprises one or more single domain antibodies (sdAbs). The sdAbs may be of the same or different origins, and of the same or different sizes. Exemplary sdAbs include, but are not limited to, heavy chain variable domains from heavy-chain only antibodies (e.g., VHH or V_{NAR}), binding molecules naturally devoid of light chains, single domains (such as V_{H} or V_{L}) derived from conventional 4-chain antibodies, humanized heavy-chain only antibodies, human single domain antibodies produced by transgenic mice or rats expressing human heavy chain segments, and engineered domains and single domain scaffolds other than those derived from antibodies. Any sdAbs known in the art or developed by the present disclosure, including the single domain antibodies described above in the present disclosure, may be used to construct the CARs described herein. The sdAbs may be derived from any species including, but not limited to mouse, rat, human, camel, llama, lamprey, fish, shark, goat, rabbit, and bovine. Single domain antibodies contemplated herein also include naturally occurring single domain antibody molecules from species other than Camelidae and sharks.

In some embodiments, the sdAb is derived from a naturally occurring single domain antigen binding molecule known as heavy chain antibody devoid of light chains (also referred herein as “heavy chain only antibodies”). Such single domain molecules are disclosed in WO 94/04678 and Hamers-Casterman, C. et al., Nature 363:446-448 (1993), for example. For clarity reasons, the variable domain derived from a heavy chain molecule naturally devoid of light chain is known herein as a VHH to distinguish it from the conventional VH of four chain immunoglobulins. Such a VHH molecule can be derived from antibodies raised in Camelidae species, for example, camel, llama, vicuna, dromedary, alpaca and guanaco. Other species besides Camelidae may produce heavy chain molecules naturally devoid of light chain, and such VHHs are within the scope of the present disclosure. In addition, humanized versions of VHHs as well as other modifications and variants are also contemplated and within the scope of the present disclosure. In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov, Protein Sci. 14:2901-2909 (2005).

In some embodiments, naturally occurring VHH domains against a particular antigen or target, can be obtained from (naïve or immune) libraries of Camelid VHH sequences. Such methods may or may not involve screening such a library using said antigen or target, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from (naïve or immune) VHH libraries may be used, such as VHH libraries obtained from (naïve or immune) VHH libraries by techniques such as random mutagenesis and/or CDR shuffling, as for example described in WO 00/43507.

In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) VH domain from a conventional 4-chain antibody by one or more of the amino acid residues that occur at the corresponding position(s) in a VHH domain of a heavy chain antibody. This can be performed in a manner known in the field, which will be clear to the skilled person. Such “camelizing” substitutions are preferably inserted at amino acid positions that form and/or are present at the VH-VL interface, and/or at the so-called Camelidae hallmark residues, as defined herein (see for example WO 94/04678, Davies and Riechmann FEBS Letters 339:285-290 (1994); Davies and Riechmann, Protein Engineering 9 (6): 531-537 (1996); Riechmann, J. Mol. Biol. 259:957-969 (1996); and Riechmann and Muyldermans, J. Immunol. Meth. 231:25-38 (1999)).

In some embodiments, the sdAb is a human single domain antibody produced by transgenic mice or rats expressing human heavy chain segments. Sec, e.g., US20090307787, U.S. Pat. No. 8,754,287, US20150289489, US20100122358, and WO2004049794.

In some embodiments, the single domain antibodies are generated from conventional four-chain antibodies. Sec, for example, EP 0 368 684; Ward et al., Nature, 341 (6242): 544-6 (1989); Holt et al., Trends Biotechnol., 21 (11): 484-490 (2003); WO 06/030220; and WO 06/003388.

In some embodiments, the extracellular antigen binding domain comprises humanized antibodies or fragment thereof. A humanized antibody can comprise human framework region and human constant region sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28 (4/5): 489-498; Studnicka et al., 1994, Protein Engineering 7 (6): 805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 93/17105, Tan et al., J. Immunol. 169:1119 25 (2002), Caldas et al., Protein Eng. 13 (5): 353-60 (2000), Morea et al., Methods 20 (3): 267 79 (2000), Baca et al., J. Biol. Chem. 272 (16): 10678-84 (1997), Roguska et al., Protein Eng. 9 (10): 895 904 (1996), Couto et al., Cancer Res. 55 (23 Supp): 5973s-5977s (1995), Couto et al., Cancer Res. 55 (8): 1717-22 (1995), Sandhu J S, Gene 150 (2): 409-10 (1994), and Pedersen et al., J. Mol. Biol. 235 (3): 959-73 (1994). See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described. Sec, e.g., Jones et al., Methods Mol Biol. 2009; 525:405-23, xiv, and De Groot et al., Cell. Immunol. 244:148-153 (2006)). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

In certain embodiments, the extracellular antigen binding domain comprises multiple binding domains. In some embodiments, the extracellular antigen binding domain comprises multispecific antibodies or fragments thereof. In other embodiments, the extracellular antigen binding domain comprises multivalent antibodies or fragments thereof. The term “specificity” refers to selective recognition of an antigen binding protein for a particular epitope of an antigen. The term “multispecific” as used herein denotes that an antigen binding protein has two or more antigen-binding sites of which at least two bind different antigens. The term “valent” as used herein denotes the presence of a specified number of binding sites in an antigen binding protein. A full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

Multispecific antibodies such as bispecific antibodies are antibodies that have binding specificities for at least two different antigens. Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

The antibodies of the present disclosure can be multivalent antibodies with two or more antigen binding sites (e.g., tetravalent antibodies), which can be readily produced by recombinant expression of nucleic acid encoding the polypeptide chains of the antibody. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In case there are multiple binding domains in the extracellular antigen binding domain of the present CARs. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. In some embodiment, a peptide linker comprises flexible residues (such as glycine and serine) so that the adjacent domains are free to move relative to each other. For example, a glycine-serine doublet can be a suitable peptide linker.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

In some embodiments, the extracellular antigen binding domain provided in the present CARs recognizes an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp 100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-1), gp 100 (Pmel 17), tyrosinase, TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### Hinge Region

In some embodiments, the CARs provided herein comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GxS)n linker, wherein x and n, independently can be an integer between 3 and 12, including 3, 4, 5, 6, 7, 8, 9, 10, 11, 12, or more.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor.

### Transmembrane Domain

The CARs of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably an eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is about 15-100 amino acids. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD1 la, CD18), ICOS (CD278), 4-1BB (CD137), GITR, CD40, BAFFR, HVEM (LIGHTR), SLAMF7, NKp80 (KLRFI), CD160, CD19, IL-2R beta, IL-2R gamma, IL-7R a, ITGA1, VLA1, CD49a, ITGA4, IA4, CD49D, ITGA6, VLA-6, CD49f, ITGAD, CD1 ld, ITGAE, CD103, ITGAL, CD1 la, LFA-1, ITGAM, CD1 lb, ITGAX, CD1 lc, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

### Intracellular Signaling Domain

The intracellular signaling domain in the CARs provided herein is responsible for activation of at least one of the normal effector functions of the immune effector cell expressing the CARs. The term “effector function” refers to a specialized function of a cell. Effector function of a NK cell, for example, may be cytolytic activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix (6-8) YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3z, FcR gamma (FCER1G), FcR beta (Fc Epsilon Rib), CD3 gamma, CD3 delta, CD3 epsilon, CD5, CD22, CD79a, CD79b, and CD66d.

### Co-Stimulatory Signaling Domain

In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. Many immune effector cells require co-stimulation, in addition to stimulation of an antigen-specific signal, to promote cell proliferation, differentiation and survival, as well as to activate effector functions of the cell.

The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as NK cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3z) and one or more co-stimulatory signaling domains. In some embodiments, the one or more co-stimulatory signaling domains and the primary intracellular signaling domain (such as cytoplasmic signaling domain of CD3z) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD32). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1 (LFA-1), CD2, CD7, LIGHT, NKG2C, B7-H3 and ligands that specially bind to CD83.

In some embodiments, the intracellular signaling domain in the CAR of the present disclosure comprises a co-stimulatory signaling domain derived from CD137 (i.e., 4-1BB). In some embodiments, the intracellular signaling domain comprises a cytoplasmic signaling domain of CDz and a co-stimulatory signaling domain of CD137. In some embodiments, the co-stimulatory signaling domains are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### Exemplary CARs

In some embodiments, the CAR provided herein comprises amino acid sequences of any one of the CARs exemplified in Section 6 below. In some embodiments, the CAR provided herein comprises amino acid sequences of any one of CARs known to those skilled in the art.

In some embodiments, the CAR provided herein comprises amino acid sequences with certain percent identity relative to any one of the CARs exemplified in Section 6 below. In some embodiments, the CAR provided herein comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of any one of the CARs exemplified in Section 6 below. In some embodiments, the CAR provided herein comprises amino acid sequences with certain percent identity relative to any one of CARs known to those skilled in the art. In some embodiments, the CAR provided herein comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of any one of CARs known to those skilled in the art.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In some embodiments, amino acid sequence modification(s) of the CARs described herein are contemplated. For example, it may be desirable to optimize the binding affinity and/or other biological properties of the extracellular domain, including but not limited to specificity, thermostability, expression level, effector functions, glycosylation, reduced immunogenicity, or solubility. Thus, in addition to the extracellular domain described herein, it is contemplated that variants of the domains described herein can be prepared. For example, scFv variants can be prepared by introducing appropriate nucleotide changes into the encoding DNA, and/or by synthesis of the desired antibody or polypeptide. Those skilled in the art who appreciate that amino acid changes may alter post-translational processes of the antibody.

Variations may be a substitution, deletion, or insertion of one or more codons encoding the polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

The antibodies generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties.

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); and (4) basic: Lys (K), Arg (R), His (H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the antibody also may be substituted, for example, with another amino acid, such as alanine or serine, to improve the oxidative stability of the molecule and to prevent aberrant crosslinking. Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions.

Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the antibody variant DNA.

Furthermore, all the above description in the present disclosure in the context of CARs also applies to engineered T cell receptor (TCR) in addition to CARs. Thus, in another aspect provided herein are method for producing immune effector cells expressing TCRs. In some embodiments, the immune effector cell is a cytotoxic cell. In certain embodiments, the cytotoxic cell is a natural killer cell. In certain embodiments, the cytotoxic cell is a cytotoxic T lymphocyte.

TCR provided herein includes recombinant or engineered TCR, which as used herein is included as a kind of functional exogenous receptor provided herein, and refers to peptide expressed into an immune cell. The functions of recombinant or engineered TCR may include for example redirecting immune activity of the immune cell against a desired type of cells, such as cancer and infected cells having specific markers at their surface. It can replace or be-co-expressed with the endogenous TCR. In some embodiments, such recombinant TCR are single-chain TCRs comprising an open reading frame where the variable Vα and Vβ domains are paired with a protein linker. This involves the molecular cloning of the TCR genes known to be specific for an antigen of choice. These chains are then introduced into T cells usually by means of a retroviral vector. Consequently, expression of the cloned TCRα and TCRβ genes endows the transduced T cell with a functional specificity determined by the pairing of these new genes. A component of a recombinant or engineered TCR is any functional subunit of a TCR, such as a recombined TCRα and TCRβ, which is encoded by an exogenous polynucleotide sequence introduced into the cell.

### 5.3.2. Polynucleotides

In certain embodiments, the disclosure provides polynucleotides that encode the CARs provided herein. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the antibody or CAR of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding CAR of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

### 5.3.3. Vectors

Also provided are vectors comprising the polynucleotides described herein. In an embodiment, the nucleic acid molecules can be incorporated into a recombinant expression vector.

The present disclosure provides vectors for cloning and expressing any one of the CARs described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human NK cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector comprises any one of the nucleic acids encoding a CAR described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid encoding the CAR is operably linked to a constitutive promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies.

In some embodiments, the nucleic acid encoding the CAR is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also contains a selectable marker gene or a reporter gene to select cells expressing the CAR from the population of host cells transfected through lentiviral vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

### 5.4. Immune Effector Cells Expressing Chimeric Antigen Receptors

In yet another aspect, provided herein are immune effector cells expressing CARs produced according to the methods provided herein, e.g., as described in Sections 4.2 and 4.3 above.

In some embodiments, the immune effector cell is a cytotoxic cell. In certain embodiments, the cytotoxic cell is a natural killer cell. In certain embodiments, the cytotoxic cell is a cytotoxic T lymphocyte.

In some embodiments, the CAR expressed in the present immune effector cells comprises an extracellular antigen binding domain; a transmembrane domain; and an intracellular signaling domain. In some embodiments, the CAR further comprises one or more additional regions/domains such as a signal peptide, hinge region, co-stimulatory signaling domain, linkers, etc., each of which can be as described in Section 4.3.1 above.

Specifically, in certain embodiments, the CARs provided herein may comprise a signal peptide at the N-terminus of the polypeptide. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor α, and IgG1 heavy chain.

The extracellular antigen binding domain of the CARs described herein comprises one or more antigen binding domains. In some embodiments, the extracellular antigen binding domain comprises an antibody or a fragment thereof. In a specific embodiment, the extracellular antigen binding domain of the present CARs comprise a single-chain Fv (sFv or scFv). In some embodiments, the extracellular antigen binding domain comprises humanized antibodies or fragment thereof.

In certain embodiments, the extracellular antigen binding domain comprises multiple binding domains. In some embodiments, the extracellular antigen binding domain comprises multispecific antibodies or fragments thereof. In other embodiments, the extracellular antigen binding domain comprises multivalent antibodies or fragments thereof. In case there are multiple binding domains in the extracellular antigen binding domain of the present CARs. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently.

In some embodiments, the extracellular antigen binding domain provided in the present CARs recognizes an antigen that acts as a cell surface marker on target cells associated with a special disease state. In some embodiments, the antigen is a tumor antigen. Tumors express a number of proteins that can serve as a target antigen for an immune response. The antigens targeted by the CAR may be antigens on a single diseased cell or antigens that are expressed on different cells that each contribute to the disease. The antigens targeted by the CAR may be directly or indirectly involved in the diseases.

Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp 100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-1), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the CARs provided herein comprise a hinge domain that is located between the extracellular antigen binding domain and the transmembrane domain. In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor.

The CARs of the present disclosure comprise a transmembrane domain that can be directly or indirectly fused to the extracellular antigen binding domain. The transmembrane domain may be derived either from a natural or from a synthetic source. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane. In some embodiments, the transmembrane domains are derived from membrane proteins of Type I, Type II or Type III. In some embodiments, the transmembrane domain of the CAR described herein is derived from a Type I single-pass membrane protein. In some embodiments, transmembrane domains from multi-pass membrane proteins may also be compatible for use in the CARs described herein. Transmembrane domains for use in the CARs described herein can also comprise at least a portion of a synthetic, non-naturally occurring protein segment. In some embodiments, the protein segment is about 15-100 amino acids. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids.

The transmembrane domain provided herein may comprise a transmembrane region and a cytoplasmic region located at the C-terminal side of the transmembrane domain. In some embodiments, the transmembrane region of the transmembrane domain comprises hydrophobic amino acid residues.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD1 la, CD18), ICOS (CD278), 4-1BB (CD137), GITR, CD40, BAFFR, HVEM (LIGHTR), SLAMF7, NKp80 (KLRFI), CD160, CD19, IL-2R beta, IL-2R gamma, IL-7R a, ITGA1, VLA1, CD49a, ITGA4, IA4, CD49D, ITGA6, VLA-6, CD49f, ITGAD, CD1 ld, ITGAE, CD103, ITGAL, CD1 la, LFA-1, ITGAM, CD1 lb, ITGAX, CD1 lc, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

The intracellular signaling domain in the CARs provided herein is responsible for activation of at least one of the normal effector functions of the immune effector cell expressing the CARs. In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. In some embodiments, the primary intracellular signaling domain contains a signaling motif known as immunoreceptor tyrosine-based activation motif, or ITAM. Exemplary ITAM-containing primary cytoplasmic signaling sequences include those derived from CD3z, FcR gamma (FCER1G), FcR beta (Fc Epsilon Rib), CD3 gamma, CD3 delta, CD3 epsilon, CD5, CD22, CD79a, CD79b, and CD66d.

In some embodiments, the CAR comprises at least one co-stimulatory signaling domain. The co-stimulatory signaling domain of the chimeric receptor described herein can be a cytoplasmic signaling domain from a co-stimulatory protein, which transduces a signal and modulates responses mediated by immune cells. In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD32) and one or more co-stimulatory signaling domains. In some embodiments, the one or more co-stimulatory signaling domains and the primary intracellular signaling domain (such as cytoplasmic signaling domain of CD3z) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD32). Multiple co-stimulatory signaling domains may provide additive or synergistic stimulatory effects.

The co-stimulatory signaling domain of any co-stimulatory molecule may be compatible for use in the CARs described herein. Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1 (LFA-1), CD2, CD7, LIGHT, NKG2C, B7-H3 and ligands that specially bind to CD83.

In some embodiments, the CAR provided herein comprises amino acid sequences of any one of the CARs exemplified in Section 6 below. In some embodiments, the CAR provided herein comprises amino acid sequences of any one of CARs known to those skilled in the art.

In some embodiments, the CAR provided herein comprises amino acid sequences with certain percent identity relative to any one of the CARs exemplified in Section 6 below. In some embodiments, the CAR provided herein comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of any one of the CARs exemplified in Section 6 below. In some embodiments, the CAR provided herein comprises amino acid sequences with certain percent identity relative to any one of CARs known to those skilled in the art. In some embodiments, the CAR provided herein comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of any one of CARs known to those skilled in the art.

### 5.5. Pharmaceutical Compositions

In one aspect, the present disclosure further provides pharmaceutical compositions comprising an engineered immune effector cell (e.g. NK cell, alpha-beta T cell, or gamma-delta T cell) expressing CARs, such as those described in Section 4.4 above. In some embodiments, a pharmaceutical composition comprises a therapeutically effective amount of the engineered immune effector cell (e.g. NK cell, alpha-beta T cell, or gamma-delta T cell) of the present disclosure and a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

### 5.6. Methods and Uses

In another aspect, provided herein are methods for using and uses of the engineered immune effector cell (e.g. NK cell) expressing CARs, such as those described in Section 4.4 above.

In some embodiments, the engineered immune effector cell (e.g. NK cell) expressing CARs provided herein are useful as allogenic CAR-NK cell therapies. In some embodiments, the present CAR-NK cell therapy has more safety features that are absent from the traditional autologous CAR-NK therapy, for example, no or low cytokine storm, no stimulation of regulatory T cells, reduced self-tissue damage, reduced induction of autoimmunity, reduced graft-versus-host disease, etc.

Such methods and uses include therapeutic methods and uses, for example, involving administration of the cells, or compositions containing the same, to a subject having a disease or disorder. In some embodiments, the cell is administered in an effective amount to effect treatment of the disease or disorder. Uses include uses of the cells in such methods and treatments, and in the preparation of a medicament in order to carry out such therapeutic methods. In some embodiments, the methods are carried out by administering the cells, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In some embodiments, the present CAR-NK cell therapies are used for treating solid tumor cancer. In other embodiments, the present CAR-NK cell therapies are used for treating blood cancer. In other embodiments, the disease or disorder is an autoimmune and inflammatory disease.

In some embodiments, the methods include administration of the cells or a composition containing the cells to a subject, tissue, or cell, such as one having, at risk for, or suspected of having the disease or disorder. In some embodiments, the cells, populations, and compositions are administered to a subject having the particular disease or disorder to be treated, e.g., via adoptive cell therapy, such as adoptive NK cell therapy. In some embodiments, the cells or compositions are administered to the subject, such as a subject having or at risk for the disease or disorder. In some embodiments, the methods thereby treat, e.g., ameliorate one or more symptom of the disease or disorder.

Methods for administration of cells for adoptive cell therapy are known, as described, e.g., in US Patent Application Publication No. 2003/0170238; U.S. Pat. No. 4,690,915; Rosenberg, Nat Rev Clin Oncol. 8 (10): 577-85 (2011); Themeli et al., Nat Biotechnol. 31 (10): 928-933 (2013); Tsukahara et al., Biochem Biophys Res Commun 438 (1): 84-9 (2013); and Davila et al., PLOS ONE 8 (4): e61338 (2013). These methods may be used in connection with the methods and compositions provided herein.

In some embodiments, the cell therapy (e.g., adoptive NK cell therapy) is carried out by autologous transfer, in which the cells are isolated and/or otherwise prepared from the subject who is to receive the cell therapy, or from a sample derived from such a subject. Thus, in some aspects, the cells are derived from a subject in need of a treatment and the cells, following isolation and processing are administered to the same subject. In other embodiments, the cell therapy (e.g., adoptive NK cell therapy) is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. The subject can be male or female and can be any suitable age, including infant, juvenile, adolescent, adult, and geriatric subjects. In some examples, the subject is a validated animal model for disease, adoptive cell therapy, and/or for assessing toxic outcomes.

The composition provided herein can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

The amount of a prophylactic or therapeutic agent provided herein that will be effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether the therapeutic agent is administered for preventive or therapeutic purposes, previous therapy, the patient's clinical history and response to the agent, and the discretion of the attending physician. The compositions, molecules and cells are in some embodiments suitably administered to the patient at one time or over a series of treatments. Multiple doses may be administered intermittently. An initial higher loading dose, followed by one or more lower doses may be administered.

In the context of genetically engineered cells, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 104, 105, 106, 107, 108, or 109 cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the compositions provided herein are administered as part of a combination treatment, such as simultaneously with or sequentially with, in any order, another therapeutic intervention, such as another antibody or engineered cell or receptor or agent, such as a cytotoxic or therapeutic agent.

In some embodiments, the compositions provided herein are co-administered with one or more additional therapeutic agents or in connection with another therapeutic intervention, cither simultaneously or sequentially in any order. In some embodiments, the cells are co-administered with another therapy sufficiently close in time such that the cell populations enhance the effect of one or more additional therapeutic agents, or vice versa. In some embodiments, the compositions provided herein are administered prior to the one or more additional therapeutic agents. In some embodiments, the compositions provided herein are administered after to the one or more additional therapeutic agents.

In certain embodiments, once the cells are administered to a mammal (e.g., a human), the biological activity of the engineered cell populations is measured by any of a number of known methods. Parameters to assess include specific binding of an engineered or natural NK cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32 (7): 689-702 (2009), and Herman et al. J. Immunological Methods, 285 (1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load.

### 5.7. Kits and Articles of Manufacture

Further provided are kits, unit dosages, and articles of manufacture comprising any of the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g., from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure.

### 6. EMBODIMENTS

The present disclosure includes the following non-limiting embodiments.

In one set of the embodiments (set A):

A1. A method for preparing an immune effector cell for transduction, comprising:
i. a first step for increasing the expression of low-density lipoprotein receptor (LDLR); and
ii. a second step for inhibiting intracellular anti-viral defense mechanisms of the immune effector cell;
wherein the first step can be conducted prior to, at the same time, or after the second step.

A2. The method of embodiment A1, wherein the immune effector cell is autologous or allogeneic.

A3. The method of embodiment A1, wherein the immune effector cell is a cytotoxic cell, and wherein optionally the cytotoxic cell is a natural killer cell.

A4. The method of any one of embodiments A1 to A3, wherein the method further comprises providing one or more transduction enhancer(s).

A5. The method of embodiment A4, wherein the transduction enhancer is Vectofusin.

A6. The method of embodiment A4, wherein the transduction enhancer is Retronectin.

A7. The method of any one of embodiments A1 to A6, wherein the method further comprises introducing a nucleic acid into the immune effector cell.

A8. The method of embodiment A7, wherein the nucleic acid comprises a promoter.

A9. The method of embodiment A8, wherein the promoter is selected from a group consisting of CAG, PGK, EF1a, and EFS promoters.

A10. The method of any one of embodiments A7 to A9, wherein the nucleic acid is delivered via a lentiviral particle.

A11. The method of embodiment A10, wherein the lentiviral particle is pseudotyped with vesicular stomatitis virus G (VSV-G).

A12. The method of any one of embodiments A7 to A11, wherein the nucleic acid encodes an exogenous functional receptor.

A13. The method of embodiment A12, wherein the exogenous functional receptor is a chimeric antigen receptor (CAR) or a TCR.

A14. The method of any one of embodiments A1 to A13, wherein the first step comprises contacting the immune effector cell with a statin.

A15. The method of embodiment A14, wherein the statin is Rosuvastatin.

A16. The method of any one of embodiments A1 to A15, wherein the second step comprises contacting the immune effector cell with an inhibitor of 3-phosphoinositide-dependent kinase 1 (PDK1).

A17. The method of embodiment A16, wherein the inhibitor of PDK1 is BX795.

In another set of embodiments (set B):

B1. A method for preparing an immune effector cell for transduction, comprising:
i. contacting the immune effector cell with a first agent that increases the expression of low-density lipoprotein receptor (LDLR);
ii. contacting the immune effector cell with a second agent that inhibits intracellular anti-viral defense mechanisms of the immune effector cell; and
iii. contacting the immune effector cell with a third agent that enhances transduction.

B2. The method of embodiment B1, wherein the immune effector cell is autologous or allogeneic.

B3. The method of embodiment B1, wherein the immune effector cell is a cytotoxic cell, and wherein optionally the cytotoxic cell is a natural killer cell.

B4. The method of any one of embodiments B1 to B3, wherein the first agent is a statin.

B5. The method of embodiment B4, wherein the statin is Rosuvastatin.

B6. The method of any one of embodiments B1 to B5, wherein the second agent is an inhibitor of 3-phosphoinositide-dependent kinase 1 (PDK1).

B7. The method of embodiment B6, wherein the inhibitor of PDK1 is BX795.

B8. The method of any one of embodiments B1 to B7, wherein the third agent is Vectofusin and/or Retronectin.

B9. The method of any one of embodiments B1 to B8, wherein the method further comprises transducing a nucleic acid into the immune effector cell.

B10. The method of embodiment B9, wherein the nucleic acid comprises a promoter.

B11. The method of embodiment B10, wherein the promoter is selected from a group consisting of CAG, PGK, EF1a, and EFS promoters.

B12. The method of any one of embodiments B9 to B11, wherein the nucleic acid is delivered via a lentiviral particle.

B13. The method of embodiment B12, wherein the lentiviral particle is pseudotyped with vesicular stomatitis virus G (VSV-G).

B14. The method of any one of embodiments B9 to B13, wherein the nucleic acid encodes an exogenous functional receptor.

B15. The method of embodiment B14, wherein the exogenous functional receptor is a chimeric antigen receptor (CAR) or a TCR.

In another set of embodiments (set C):

C1. A method comprising:
i. contacting an immune effector cell with a first agent that increases the expression of low-density lipoprotein receptor (LDLR);
ii. contacting the immune effector cell with a second agent that inhibits intracellular anti-viral defense mechanisms of the immune effector cell;
iii. contacting the immune effector cell with a third agent that enhances transduction; and
iv. introducing a nucleic acid into the immune effector cell.

C2. The method of embodiment C1, wherein the immune effector cell is autologous or allogeneic.

C3. The method of embodiment C1, wherein the immune effector cell is a cytotoxic cell, and wherein optionally the cytotoxic cell is a natural killer cell.

C4. The method of any one of embodiments C1 to C3, wherein the first agent is a statin.

C5. The method of embodiment C2, wherein the statin is Rosuvastatin.

C6. The method of any one of embodiments C1 to C5, wherein the second agent is an inhibitor of 3-phosphoinositide-dependent kinase 1 (PDK1).

C7. The method of embodiment C6, wherein the inhibitor of PDK1 is BX795.

C8. The method of any one of embodiments C1 to C7, wherein the third agent is Vectofusin and/or Retronectin.

C9. The method of any one of embodiments C1 to C8, wherein the nucleic acid comprises a promoter.

C10. The method of embodiment C9, wherein the promoter is selected from a group consisting of CAG, PGK, EF1a, and EFS promoters.

C11. The method of any one of embodiments C1 to C10, wherein the nucleic acid is delivered via a lentiviral particle.

C12. The method of embodiment C11, wherein the lentiviral particle is pseudotyped with vesicular stomatitis virus G (VSV-G).

C13. The method of any one of embodiments C1 to C12, wherein the nucleic acid encodes an exogenous functional receptor.

C14. The method of embodiment C13, wherein the exogenous functional receptor is a chimeric antigen receptor (CAR) or a TCR.

In another set of embodiments (set D):

D1. A method for preparing an immune effector cell for transduction, comprising a step for increasing the expression of low-density lipoprotein receptor (LDLR).

D2. The method of embodiment D1, wherein the immune effector cell is autologous or allogeneic.

D3. The method of embodiment D1, wherein the immune effector cell is a cytotoxic cell, and wherein optionally the cytotoxic cell is a natural killer cell.

D4. The method of embodiment D1, wherein the immune effector cell is a gamma delta T cell.

D5. The method of embodiment D1, wherein the immune effector cell is an alpha beta T cell.

D6. The method of embodiment D1, wherein the step for increasing the expression of low-density lipoprotein receptor (LDLR) comprises contacting the immune effector cell with a statin.

D7. The method of embodiment D6, wherein the statin is Rosuvastatin.

D8. The method of embodiment D6, wherein the statin is Atorvastatin.

D9. The method of any one of embodiment D1 to D8, further comprising a step for inhibiting intracellular anti-viral defense mechanisms of the immune effector cell.

D10. The method of embodiment D9, wherein the step for inhibiting intracellular anti-viral defense mechanisms of the immune effector cell, comprising contacting the immune effector cell with an inhibitor of 3-phosphoinositide-dependent kinase 1 (PDK1).

D11. The method of embodiment D10, wherein the inhibitor of PDK1 is BX795.

D12. The method of any one of embodiment D1 to D11, wherein the method further comprises providing one or more transduction enhancer(s).

D13. The method of embodiment D4, wherein the transduction enhancer is

### Vectofusin.

D14. The method of embodiment D4, wherein the transduction enhancer is Retronectin.

D15. The method of any one of embodiment D1 to D14, wherein the method further comprises introducing a nucleic acid into the immune effector cell.

D16. The method of embodiment D15, wherein the nucleic acid comprises a promoter.

D17. The method of embodiment D16, wherein the promoter is selected from a group consisting of CAG, PGK, EF1a, and EFS promoters.

D18. The method of any one of embodiment D15 to D17, wherein the nucleic acid is delivered via a lentiviral particle.

D19. The method of embodiment D18, wherein the lentiviral particle is pseudotyped with vesicular stomatitis virus G (VSV-G).

D20. The method of any one of embodiment D15 to D19, wherein the nucleic acid encodes an exogenous functional receptor.

D21. The method of embodiment D20, wherein the exogenous functional receptor is a chimeric antigen receptor (CAR) or a TCR.

In another set of embodiments (set E):

E1. A method for preparing an immune effector cell for transduction, comprising contacting the immune effector cell with an agent that increases the expression of low-density lipoprotein receptor (LDLR).

E2. The method of embodiment E1, wherein the immune effector cell is autologous or allogeneic.

E3. The method of embodiment E1, wherein the immune effector cell is a cytotoxic cell, and wherein optionally the cytotoxic cell is a natural killer cell.

E4. The method of embodiment E1, wherein the immune effector cell is a gamma delta T cell.

E5. The method of embodiment E1, wherein the immune effector cell is an alpha beta T cell.

E6. The method of embodiment E1, wherein the step for increasing the expression of low-density lipoprotein receptor (LDLR) comprises contacting the immune effector cell with a statin.

E7. The method of embodiment E6, wherein the statin is Rosuvastatin.

E8. The method of embodiment E6, wherein the statin is Atorvastatin.

E9. The method of any one of embodiments E1 to E8, further comprising a step for inhibiting intracellular anti-viral defense mechanisms of the immune effector cell.

E10. The method of embodiment E9, wherein the step for inhibiting intracellular anti-viral defense mechanisms of the immune effector cell, comprising contacting the immune effector cell with an inhibitor of 3-phosphoinositide-dependent kinase 1 (PDK1).

E11. The method of embodiment E10, wherein the inhibitor of PDK1 is BX795.

E12. The method of any one of embodiments E1 to E11, wherein the method further comprises providing one or more transduction enhancer(s).

E13. The method of embodiment E12, wherein the transduction enhancer is Vectofusin.

E14. The method of embodiment E12, wherein the transduction enhancer is Retronectin.

E15 The method of any one of embodiments E1 to E14, wherein the method further comprises introducing a nucleic acid into the immune effector cell.

E16. The method of embodiment E15, wherein the nucleic acid comprises a promoter.

E17 The method of embodiment E16, wherein the promoter is selected from a group consisting of CAG, PGK, EF1a, and EFS promoters.

E18. The method of any one of embodiments E15 to E17, wherein the nucleic acid is delivered via a lentiviral particle.

E19. The method of embodiment E18, wherein the lentiviral particle is pseudotyped with vesicular stomatitis virus G (VSV-G).

E20. The method of any one of embodiments E15 to 17, wherein the nucleic acid encodes an exogenous functional receptor.

E21. The method of embodiment E20, wherein the exogenous functional receptor is a chimeric antigen receptor (CAR) or a TCR.

### 7. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 7.1. Example 1—Generation of Natural Killer CAR (CAR-NK) Cells

NK cells were first activated in preparation of the transduction. One day 0, 0.5-1×10⁶ NK cells per well were distributed in a 24-well plate.

Loaded Anti-Biotin MACSiBead particles (Miltenyi Biotech, Cat #130-094-483) for NK cell activation were resuspended thoroughly and were transferred to a suitable tube at the amount of 5 μL (5×10⁵ loaded Anti-Biotin MACSiBead particles) per 1×10⁶ NK cells. 100 μL culture medium was added to the loaded Anti-Biotin MACSiBead particles. The mixture was centrifuged at 300×g for 5 minutes so that the beads were washed prior to being added to the plated NK cells. The supernatant was aspirated and the loaded Anti-Biotin MACSiBead Particles were resuspended in 5-10 μL of fresh NK MACS medium with NK MACS supplement (Miltenyi Biotech, Cat #130-114-429). The prepared Anti-Biotin MACSiBead particles were added to the plated NK cells and were mixed gently by pipetting up and down 2-3 times. The cells were then incubated at 37° C. and 5% CO₂.

Lentiviral transduction was performed on day 5 or 6. NK cells were resuspended to determine if cell number had changed from day 0. Lentiviral particles were thawed on ice. Once thawed, lentiviral particles were taken out from ice and equilibrated to room temperature before being added to the activated NK cells. The amount of virus needed for transducing cells was determined based on desired multiplicity of infection (MOI). Once the NK cells were transduced with lentiviral particles, they were returned to the incubator at 37° C. and 5% CO₂.

Cells were monitored as early as day 3-4 post transduction for surface CAR expression. The cell number of NK cells was determined. 1×10⁵ NK cells were collected from each sample and transferred into wells of a microtiter plate. The cells in each well of the microtiter plate were washed twice with 200 μL FACS Buffer each time. The wells were stained with 100 μL staining buffer containing appropriate CAR detection antibodies. The plate was incubated for at least 30 minutes at 4° C. without light. The cells were then washed twice with 150 μL FACS Buffer each time and were centrifuged at 400×g for 4 minutes at room temperature. The CAR expression on NK cells was then measured using flow cytometry analysis.

To evaluate CAR transduction efficiency and surface CAR stability, NK-like cell lines (e.g., NK-L) and primary NK cells were transduced with a Antigen-1 CAR at a MOI of 5. Surface Antigen-1 CAR expression was monitored at multiple time points post transduction using an anti-myc tag AF647 antibody (Cell Signaling, Cat #2233, 1:200).

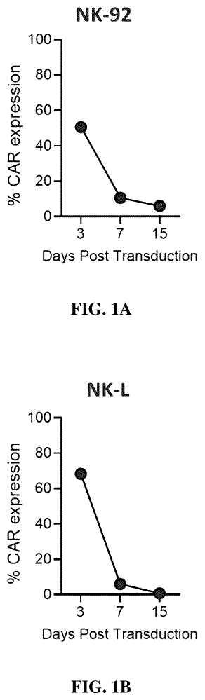

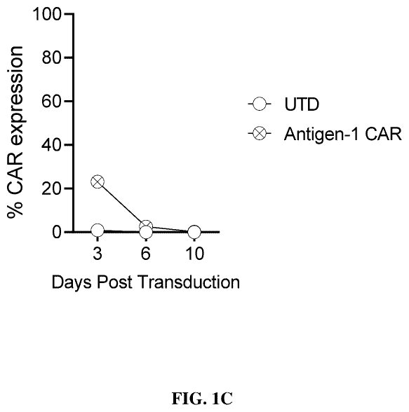

As shown in FIGS. 1A-1C, NK-like cell lines (NK-92 and NK-L) were transduced efficiently at early time points (day 3), compared to low expression seen in primary NK cells. However, Antigen-1 CAR expression was downregulated over time in both NK-like cell lines (NK-92 and NK-L) and primary NK cells.

### 7.2. Example 2—Optimization of Gene Transfer

To improve CAR transduction efficiency and surface CAR stability, the following additional steps were implemented before or at the same time of adding the lentiviral particles to the cells.

### 7.2.1. Inhibitors of 3-Phosphoinositide-Dependent Kinase 1 (PDK1)

NK cells are evolutionarily selected to have resistance against viral infection. PDK1 inhibitors can block pattern recognition receptors that are innately used by immune cells such as NK cells during viral infection.

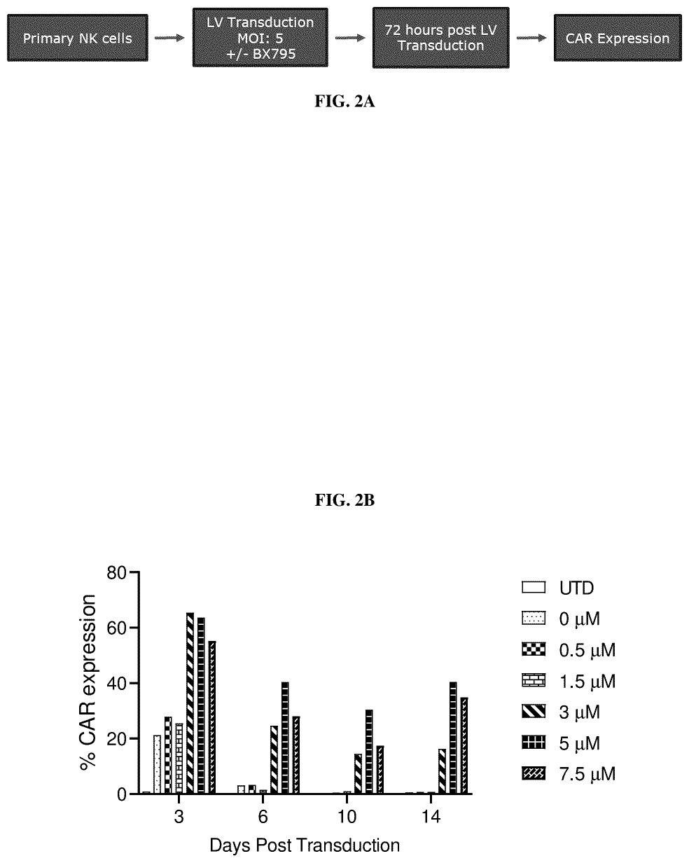

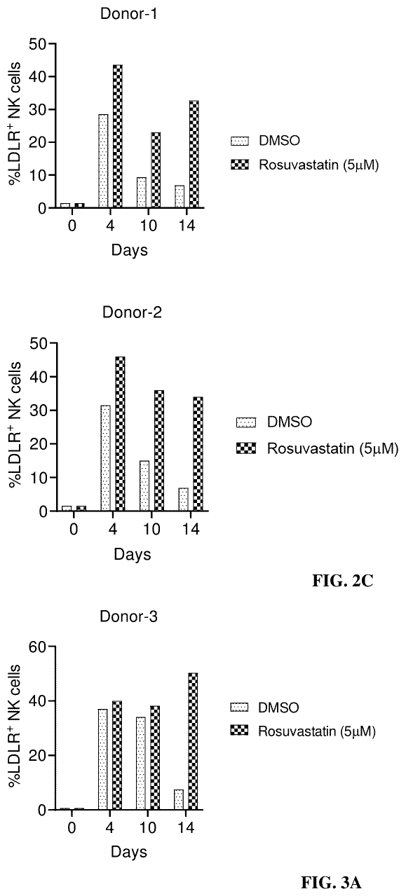

To test whether PDK1 inhibitors can improve CAR transduction efficiency or surface CAR stability, primary NK cells were activated and transduced with a Antigen-1 CAR in the presence or absence of an PDK1 inhibitor, BX795 (InvivoGen, cat #tlrl-bx7), at concentrations varying from 0.5 to 7.5 μM. BX795 was incorporated into the transduction process by being added to the cells with the viral supernatants and mixed gently. Surface CAR expression was monitored at regular intervals using an anti-myc tag AF647 antibody (Cell Signaling, Cat #2233, 1:200). The schematic schedule of the experiment is shown in FIG. 2A.

As shown in FIGS. 2B-2C, compared to cells transduced without any BX795, primary NK cells transduced with BX795 showed improved surface CAR stability in a dose dependent manner (n=2).

### 7.2.2. Statins

For the lentiviral pseudotype vesicular stomatitis virus G (VSV-G), low-density lipoprotein receptor (LDLR) serves as a main entry receptor during internalization. Statins are commonly used to upregulate the expression of LDLR.

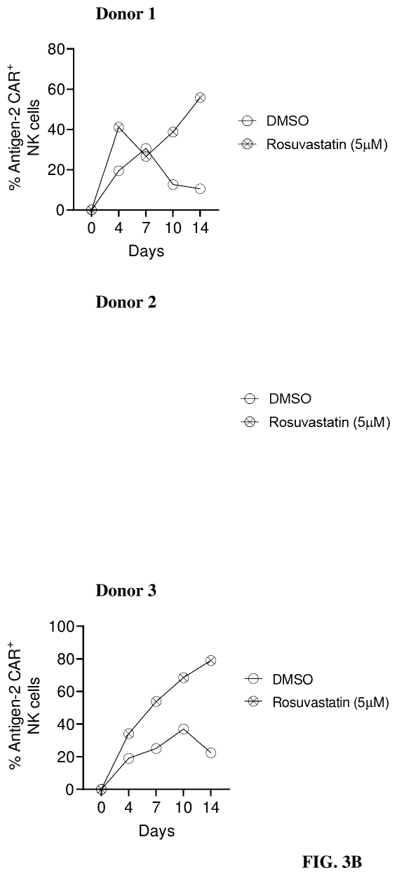

To test whether statins can improve CAR transduction efficiency or surface CAR stability, cells were pre-treated with 5 μM Rosuvastatin (Cayman Chemical, cat #12029) during the activation prior to the transduction. Then, activated primary NK cells were transduced with a Antigen-2 CAR at MOI of 5. Cells pre-treated with DMSO were included as a negative control. As shown in FIG. 3A, incubation of primary NK cells with Rosuvastatin during the activation led to increased levels of LDLR expression on the cells (n=3). As shown in FIG. 3B, the increased levels of LDLR expression on primary NK cells was associated with increased Antigen-2 CAR expression.

### 7.2.3. Transduction Enhancers

Retronectin is a recombinant human fibronectin fragment that enhances lentiviral- and retroviral-mediated gene transduction by aiding the colocalization of target cells and viral particles. Vectofusin is a fully synthetic non-toxic cationic amphipathic peptide with viral transduction enhancing capacity.

To test whether transduction enhancers can improve CAR transduction efficiency or surface CAR stability, primary NK cells were activated and transduced in the presence or absence of Retronectin and Vectofusin.

The day before plating primary NK cells, Retronectin (Takara Bio Inc., Cat #T202) was diluted in sterile PBS from the 1 μg/μL stock to make a 20 μg/mL working solution. The Retronectin working solution was added to the wells and was evenly spread inside the wells. The plate was kept at 4° C. overnight before the day of the plating. Retronectin was removed from the wells after the overnight incubation. PBS with 2% BSA was added to each well. The plate was incubated at room temperature for 30 minutes and was washed twice with PBS. NK cells were then plated and activated as described in Section 6.1 above.

Activated primary NK cells were transduced with a Antigen-2 CAR at MOI of 5. Vectofucin (Miltenyi, cat #130-111-163) with a working concentration of 10 μg/mL in total culture volume was mixed with viral supernatants and was incubated on ice for 10-15 minutes. The mixture was equilibrated to room temperature before being added to NK cells and was mixed with cell suspension by being pipetted up and down. Then, the cells were incubated at 37° C. and 5% CO₂. The CAR expression in primary NK cells were evaluated at day 10.

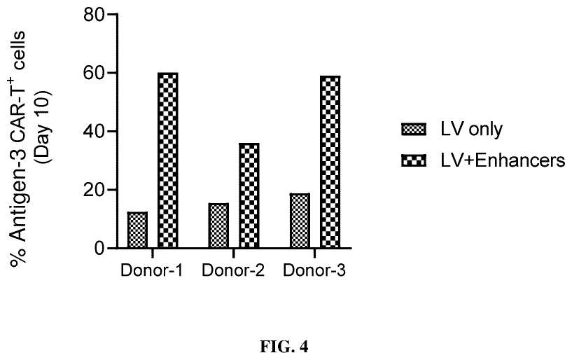

As shown in FIG. 4, the presence of transduction enhancers, Retronectin and Vectofusin, during the lentiviral transduction of primary NK cells was associated with increased Antigen-3 CAR expression (n=3).

### 7.2.4. Combination

To address the challenge of lentiviral transduction in primary NK cells, primary NK cells were transduced with a combination treatment of PDK1 inhibitors, statins, and transduction enhancers. Activated NK cells were transduced with a Antigen-3 CAR at a MOI of 5. Cells were transduced in a Retronectin-coated dish along with BX795, Rosuvastatin, and Vectofucin. The approaches of incorporating BX795, Rosuvastatin, and Vectofucin were the same as described above under Section 6.2. After the transduction, the plate was spun down for 2 hours at 850×g at 37° C. Once the spin was done, the plate was returned back into the incubator at 37° C. and 5% CO₂. CAR expression was monitored with an anti-Antigen-3 CAR recombinant protein AF647 antibody (2 μg/mL) at different time points post transduction.

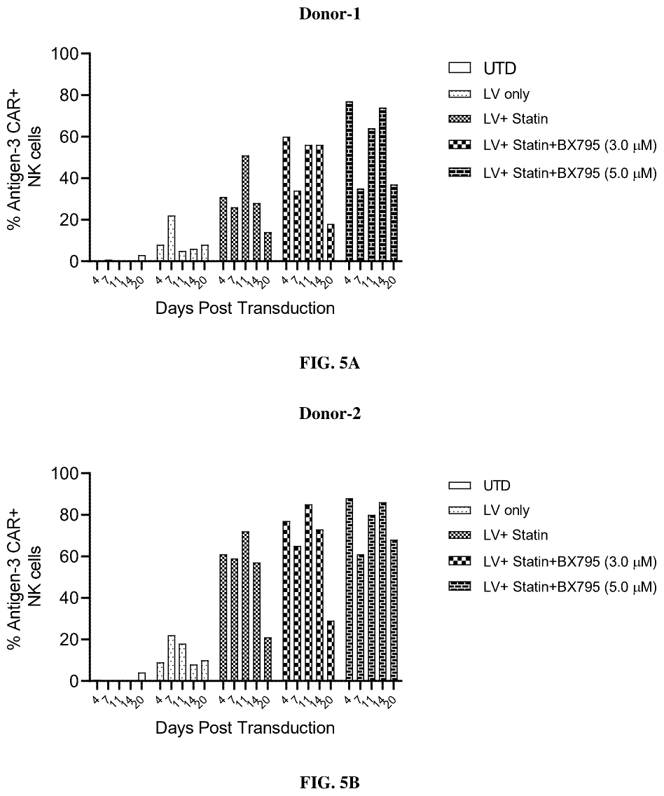

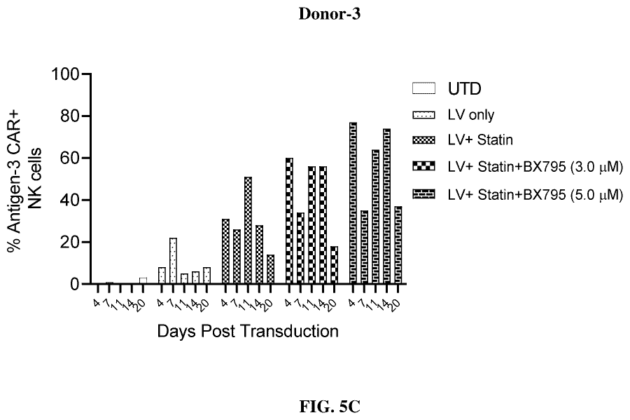

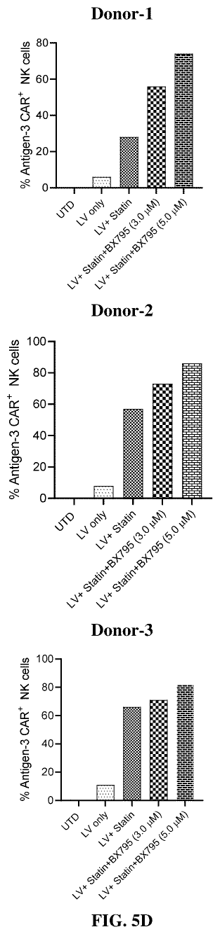

As shown in FIGS. 5A-5D, compared to lentivirus alone, primary NK cells transduced with a combination treatment of BX795, Rosuvastatin, and Vectofucin displayed higher expression and stability of surface CAR (n=3).

To address the challenge of lentiviral transduction in primary T cells, primary αβ T cells were transduced with a combination treatment of PDK1 inhibitors, statins, and transduction enhancers. Activated αβ T cells were transduced with a Antigen-3 CAR (internal) at a MOI of 5. Cells were transduced in a Retronectin-coated dish along with BX795 (3 μM), Rosuvastatin (1 μM), and Vectofucin. The approaches of incorporating BX795, Rosuvastatin, and Vectofucin were the same as described above under Section 6.2. After the transduction, the plate was spun down for 2 hours at 850×g at 37° C. Once the spin was done, the plate was returned back into the incubator at 37° C. and 5% CO₂. CAR expression was monitored with an anti-Antigen-3 CAR recombinant protein AF647 antibody (2 μg/mL) at different time points post transduction.

As shown in FIG. 6, compared to lentivirus alone, primary αβ T cells transduced with a combination treatment of BX795, Rosuvastatin, Retronectin, and Vectofucin displayed higher CAR expression (n=3).

### 7.3. Example 3-Optimization of Gene Transfer in γδ T Cells

Lentiviral transduction of primary γδ T cells, using VSV-G pseudotyped lentiviral vectors, has been associated with low levels of CAR expression, limiting CAR screening capabilities. Lentiviral transduction of primary γδ T cells in the presence of compounds targeting LDLR (Statin), anti-viral pathway (BX795), and vectofusin was evaluated.

Four primary γδ T cell samples from healthy donors were expanded with Zoledronic acid (ZOL) for five days, transduced in the presence or absence of enhancers (rosuvastatin, BX795, and Vectofusin), and evaluated for CAR expression. Briefly, frozen PBMC cells were thawed and resuspended at 1E6 cells/ml. PBMC cells were activated with Zoledronic acid, 10 μM and expanded in RPMI medium in the presence of IL-2 (100IU) and IL-15 (10 ng). On Day 6, cells were transduced with the lentiviral vector encoding the CAR specific to Antigen-4 lentiviral vector at a MOI of 5, including the following steps: (1) Antigen-4-specific CAR-encoding lentiviral vectors were pre-incubated with statin, vectofusin-1 (10 μg/ml) on ice for 10 mins prior to adding to the cells; (2) BX795 at 5 μM were added to cells and mixed gently; (3) cells were spinoculated at 850 g for 2 hrs at 32° C.; and (4) cells were returned to the incubator at 37° C. Cells were analyzed for CAR expression by flow cytometry at Days 3, 6 and 10.

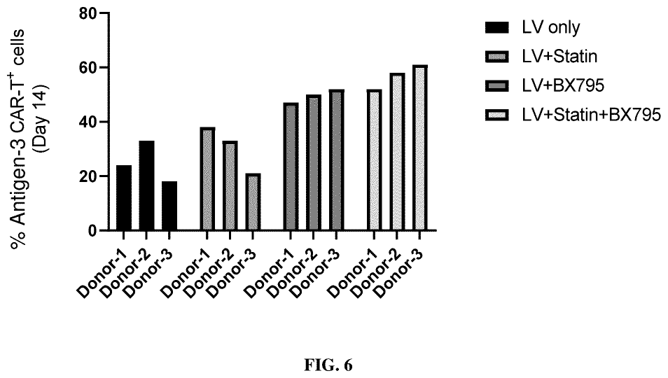

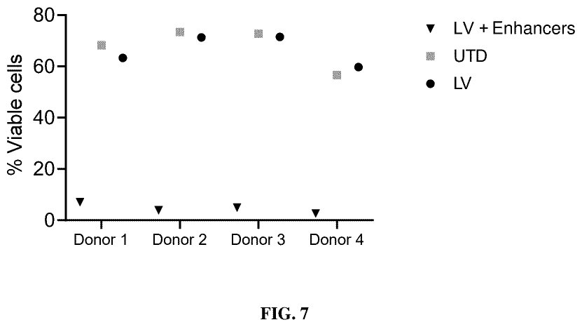

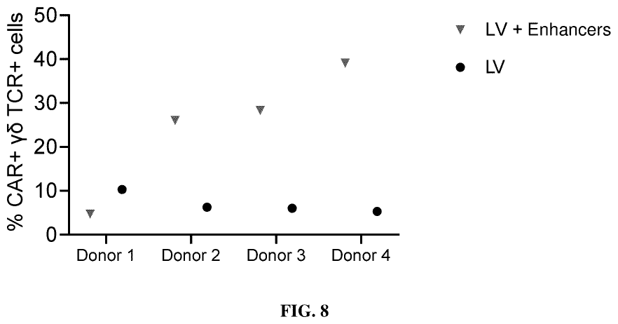

Lentiviral transduction of primary γδ T cells in the presence of enhancers (rosuvastatin, BX795, and Vectofusin) decreased cell viability, as compared to the lentiviral transduction without the enhancers (FIG. 7). Lentiviral transduction of primary γδ T cells in the presence of enhancers increased the CAR levels of primary γδ T cells as compared to lentiviral vector only (FIG. 8).

Conclusion: although the lentiviral transduction of primary γδ T cells in the presence of enhancers (rosuvastatin, BX795, and Vectofusin) increased transduction, it was also associated with increased cell death, thus making practical implementation of this protocol challenging.

Next, the lentiviral transduction of primary γδ T cells, using VSV-G pseudotyped lentiviral vectors, in the presence of enhancers, targeting LDLR, was evaluated.

Three primary γδ T cell samples from healthy donors were expanded with Zoledronic acid (ZOL) for five days, transduced using a lentiviral vector encoding Antigen-4-specific CAR, in the presence or absence of Rosuvastatin and Atorvastatin at different concentrations, and evaluated for CAR expression. Briefly, frozen PBMC cells were thawed and resuspended at 1E6 cells/ml. PBMC cells were activated with Zoledronic acid, 10 μM and expanded in RPMI medium in the presence of IL-2 (100IU) and IL-15 (10 ng). On Day 6, cells were transduced with lentiviral vector encoding the Antigen-4 specific CAR at a MOI of 5, including the following steps: (1) Antigen-4 CAR encoding lentiviral vectors were pre-incubated with statin on ice for 10 mins prior to adding to the cells; (2) cells were spinoculated at 850 g for 2 hrs at 32° C.; and (3) cells were returned to the incubator at 37° C. Cells were analyzed for CAR expression by flow cytometry at Days 3, 6 and 10.

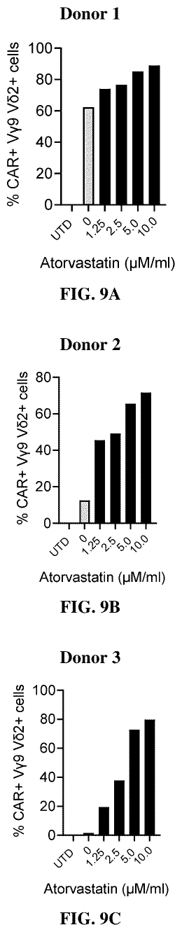

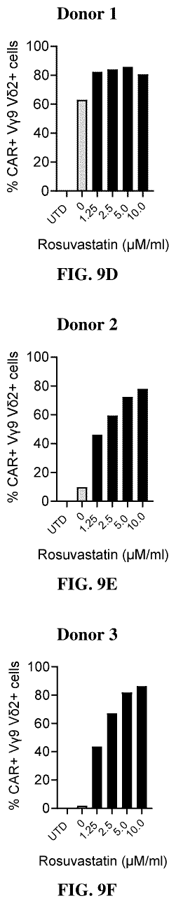

Lentiviral transduction of primary γδ T cells in the presence of statin was associated with the higher CAR expression, as compared to lentiviral vector only-transduced cells (FIGS. 9A-9F). This novel protocol has also achieved increased CAR expression on primary γδ T cells (FIGS. 9A-9F), enabling CAR screening for future experimentations.

Next, the effect of pre-treatment with statin in lentiviral transduction of primary γδ T cells on LDLR expression was evaluated. Three primary γδ T cells samples from healthy donors were expanded with Zol for five days, pre-treated in the presence or absence of Rosuvastatin and Atorvastatin at different concentrations, as described above, and evaluated for LDLR expression.

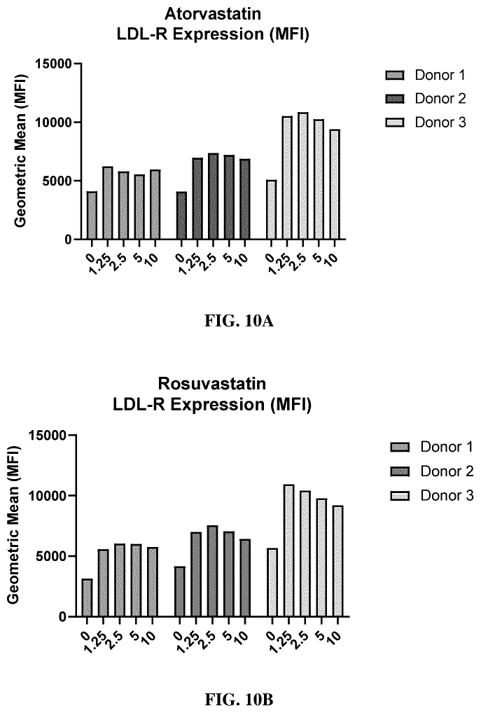

Pre-treatment of primary γδ T cells with statin, either Atorvastatin (FIG. 10A) or Rosuvastatin (FIG. 10B) increased the LDLR expression as compared to lentiviral vector only-transduced cells.

Next, the effect of pre-treatment with statin in lentiviral transduction of primary γδ T cells on the frequency of Vγ9Vδ2 T cell subset within the γδ T cells was evaluated. Three primary γδ T cells samples from healthy donors were expanded with Zol for five days, pre-treated in the presence or absence of Rosuvastatin or Atorvastatin at different concentrations, as described above, and evaluated for Vγ9Vδ2 expression, using flow cytometry.

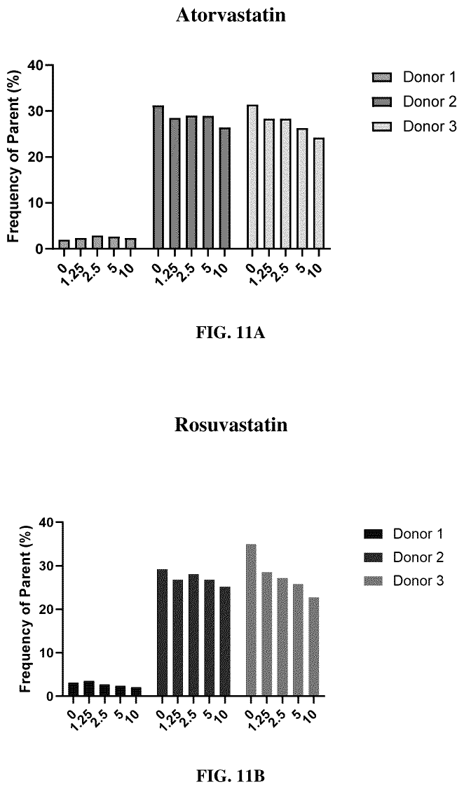

Pre-treatment of primary γδ T cells with statin, either Atorvastatin (FIG. 11A) or Rosuvastatin (FIG. 11B) did not change the frequency of Vγ9Vδ2 cells as compared to lentiviral vector only-transduced cells at 0 statin concentration.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

1. A method for preparing an immune effector cell for transduction, comprising a step for increasing the expression of low-density lipoprotein receptor (LDLR).

2. The method of claim 1, wherein the immune effector cell is autologous or allogeneic.

3. (canceled)

4. The method of claim 1, wherein the immune effector cell is a gamma delta T cell or an alpha beta T cell.

5. (canceled)

6. The method of claim 1, wherein the step for increasing the expression of low-density lipoprotein receptor (LDLR) comprises contacting the immune effector cell with a statin.

7. The method of claim Error! Reference source not found.6, wherein the statin is Rosuvastatin or Atorvastatin.

8. (canceled)

9. The method of claim 1, further comprising a step for inhibiting intracellular anti-viral defense mechanisms of the immune effector cell.

10. The method of claim Error! Reference source not found.9, wherein the step for inhibiting intracellular anti-viral defense mechanisms of the immune effector cell, comprising contacting the immune effector cell with an inhibitor of 3-phosphoinositide-dependent kinase 1 (PDK1).

11. The method of claim 10, wherein the inhibitor of PDK1 is BX795.

12. The method of claim 1, wherein the method further comprises providing one or more transduction enhancer(s).

13. The method of claim 12, wherein the transduction enhancer is Vectofusin or Retronectin.

14. (canceled)

15. The method of claim 1, wherein the method further comprises introducing a nucleic acid into the immune effector cell.

16. The method of claim 15, wherein the nucleic acid comprises a promoter.

17. (canceled)

18. The method of claim 15, wherein the nucleic acid is delivered via a lentiviral particle.

19. The method of claim 18, wherein the lentiviral particle is pseudotyped with vesicular stomatitis virus G (VSV-G).

20. The method of claim 15, wherein the nucleic acid encodes an exogenous functional receptor that is a chimeric antigen receptor (CAR) or a TCR.

21. (canceled)

22. A method for preparing an immune effector cell for transduction, comprising contacting the immune effector cell with an agent that increases the expression of low-density lipoprotein receptor (LDLR).

23-26. (canceled)

27. The method of claim 22, wherein the step for increasing the expression of low-density lipoprotein receptor (LDLR) comprises contacting the immune effector cell with a statin.

28-29. (canceled)

30. The method of claim 22, wherein the method further comprising inhibiting intracellular anti-viral defense mechanisms of the immune effector cell.

31-32. (canceled)

33. The method of claim 22, wherein the method further comprises providing one or more transduction enhancer(s).

34-35. (canceled)

36. The method of claim 22, wherein the method further comprises introducing a nucleic acid into the immune effector cell.

37-42. (canceled)
